(19) United States
(12) Patent Application Publication
(10) Pub. No.: US 2026/0212999 A1
(43) Pub. Date: Jul. 23, 2026

(54) SYSTEM AND METHOD FOR PROCESSING ULTRASOUND IMAGES

(71) Applicants: New York University, New York, NY (US); Yeda Research And Development Co. Ltd., Rehovot (IL)

(72) Inventors: Itay KEZURER, Rehovot (IL); Yoram ESHEL, Netanya (IL); Achiau LUDOMIRSKY, New York, NY (US); Yaron LIPMAN, Netzer Sereni (IL)

(21) Appl. No.: 19/557,236

(22) Filed: Mar. 5, 2026

Related U.S. Application Data
(62) Division of application No. 19/344,654, filed on Sep. 30, 2025.
Application No. 19/344,654 is a division of application No. 18/060,584, filed on Dec. 1, 2022, now Pat. No. 12,450,483.
Application No. 18/060,584 is a continuation of application No. 16/412,675, filed on May 15, 2019, now Pat. No. 11,593,638.
(60) Provisional application No. 62/671,692, filed on May 15, 2018.

(51) Int. Cl. G16H 30/40; A61B 5/00; A61B 8/00; A61B 8/13; G06N 3/08; G06T 3/20; G06T 3/60; G06T 7/00; G06T 12/00; G09B 23/28

(52) U.S. Cl. CPC G16H 30/40; A61B 5/7267; A61B 8/13; A61B 8/4254; A61B 8/4427; A61B 8/463; G06N 3/08; G06T 3/20; G06T 3/60; G06T 7/0012; G06T 12/00; G09B 23/286; G06T 2207/10132; G06T 2207/20081; G06T 2207/20084; G06T 2207/30048

(57) ABSTRACT

A computer-implemented method provides real-time visual guidance for orienting an ultrasound probe toward a canonical ultrasound view. The method involves receiving successive ultrasound images acquired by the probe during a scanning procedure. A trained neural network generates a spatial mapping from the received images without reliance on external position or motion sensors. This mapping encodes adjustments in probe position and rotation needed to achieve a target spatial pose associated with the canonical view. The spatial mapping is then transformed into visual guidance data. One or more visual guidance elements, derived from this data, are generated and displayed on a graphical user interface to instruct a user on how to manipulate the probe. The visual guidance elements are continuously updated until the canonical ultrasound view is achieved, helping non-experts capture high-quality diagnostic images.

### CROSS REFERENCE TO RELATED APPLICATIONS

This application is a divisional application of U.S. Ser. No. 19/344,654, filed Dec. 1, 2022, which is a divisional application of U.S. Ser. No. 18/060,584, filed Dec. 1, 2022, now issued as U.S. Pat. No. 12,450,483, which is a continuation application of U.S. patent application Ser. No. 16/412,675, filed May 15, 2019, now issued as U.S. Pat. No. 11,593,638 on Feb. 28, 2023, which claims priority from U.S. provisional patent application 62/671,692, filed May 15, 2018, all of which are incorporated herein by reference.

### FIELD OF THE INVENTION

The present invention relates to mobile handheld ultrasound machines generally and to orientation for correct use in particular.

### BACKGROUND OF THE INVENTION

A medical ultrasound (also known as diagnostic sonography or ultrasonography) is a diagnostic imaging technique based on the application of an ultrasound. It is used to create an image of internal body structures such as tendons, muscles, joints, blood vessels and internal organs.

Acquiring accurate images in order to perform an effective examination and diagnosis requires placing the ultrasound transducer in an angular position in space with the pertinent organ or body part, as is illustrated in FIG. 1 to which reference is now made. FIG. 1 shows an ultrasound image of an organ of interest 12 taken with a transducer 14. It will be appreciated that the art of navigating transducer 14 to the exact angular position required to achieve the optimal or “canonical” image of organ 12 is crucial to the success of the ultrasound examination. The process typically requires a trained and skilled sonographer.

For example, in order to perform an echocardiogram, the sonographer has to take images of the heart from various canonical directions, such as four-chamber and two-chamber views. The correct positioning of the transducer is crucial to receiving the optimal view of the left ventricle and consequently to extract the functional information of the heart.

Mobile ultrasound machines or devices are known in the art, such as the Lumify commercially available from Philips. These mobile ultrasound machines are available in the form of a transducer that communicates with a program downloadable to any portable handheld device such as a smart phone or a tablet.

The availability of such devices means that ultrasounds may be performed off-site (away from hospitals, etc.) for example, as a triage tool for ambulances or even in the battlefield, at urgent care facilities, nursing homes, etc. without requiring bulky expensive equipment.

### SUMMARY OF THE PRESENT INVENTION

There is therefore provided, in accordance with a preferred embodiment of the present invention, a computer-implemented method for providing real-time visual guidance for orienting an ultrasound probe toward a canonical ultrasound view. The method includes receiving successive ultrasound images acquired by an ultrasound probe during a scanning procedure, each ultrasound image reflecting a current probe position and rotation relative to an anatomical region. The method further includes generating, by a trained neural network, from the received ultrasound images, without reliance on external position or motion sensors, a spatial mapping which encodes one or more adjustments in probe position and rotation required to achieve a target spatial pose associated with the canonical ultrasound view, transforming the spatial mapping into visual guidance data to achieve the required adjustment, generating and displaying, on a graphical user interface, one or more visual guidance elements derived from the visual guidance data to instruct a user how to manipulate the probe, and updating the visual guidance elements until the canonical ultrasound view is achieved.

Moreover, in accordance with a preferred embodiment of the present invention, the one or more visual guidance elements include a set of arrows, and where the type of rotation visually communicated is one of a pitch or a yaw.

Further, in accordance with a preferred embodiment of the present invention, the one or more visual guidance elements includes colored rotation markings.

Still further, in accordance with a preferred embodiment of the present invention, the updating is performed until the current probe orientation is within a predefined error range of the target probe orientation.

Additionally, in accordance with a preferred embodiment of the present invention, the graphical user interface provides an instruction to the user to either move the ultrasound probe or to continue viewing at the current probe orientation.

Moreover, in accordance with a preferred embodiment of the present invention, the updating step generates updated visual guidance for each new subsequent ultrasound image captured by the probe.

Further, in accordance with a preferred embodiment of the present invention, the trained orientation neural network was trained using a training dataset including at least one canonical view, a plurality of non-canonical images captured around the at least one canonical view, and corresponding ground-truth transformations, each ground-truth corrective orientation representing a required adjustment in position and rotation between a non-canonical image and the canonical view.

Still further, in accordance with a preferred embodiment of the present invention, the ground-truth corrective orientations were determined by from inertia measurement unit (IMU) data received from an IMU mounted on a training probe during a training session.

Additionally, in accordance with a preferred embodiment of the present invention, the training includes minimizing a loss function, the loss function configured to reduce a distance between a calculated corrective orientation produced by the orientation neural network and the corresponding ground-truth corrective orientation for each non-canonical image.

Moreover, in accordance with a preferred embodiment of the present invention, the loss function further includes an additional term based on a probability measure to constrain the calculated transformation relative to known motions between a plurality of different canonical views.

Further, in accordance with a preferred embodiment of the present invention, the canonical view is one of a plurality of available canonical views for a body part.

Still further, in accordance with a preferred embodiment of the present invention, the method further includes, upon the current probe orientation reaching the target probe orientation, making a diagnosis from the current ultrasound image.

Additionally, in accordance with a preferred embodiment of the present invention, the computing device is one of: a smartphone, a tablet, a laptop, a personal computer, or a smart appliance.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, images, and advantages thereof, may best be understood by reference to the following detailed description when read with the accompanying drawings in which:

FIG. 1 is a schematic illustration of how an ultrasound transducer is placed to capture an image of a body part;

FIG. 2 is a schematic illustration of an ultrasound navigator; constructed and operative in accordance with the present invention;

FIGS. 3A and 3B are schematic illustrations of how the navigator of FIG. 2 may aid a non-sonographer orientate probe a transducer in order to capture a suitable image of a body part, constructed and operative in accordance with the present invention;

FIG. 4 is a schematic illustration of the transformation between the orientation of a training probe for a non-canonical image of an organ and its associated canonical image, constructed and operative in accordance with the present invention;

FIG. 5 is a schematic illustration of the training process for an orientation neural network, constructed and operative in accordance with the present invention;

FIG. 6 is a schematic illustration of the elements of the navigator of FIG. 2, constructed and operative in accordance with the present invention;

FIG. 7 is a schematic illustration of the elements of an alternative embodiment to the navigator of FIG. 2, constructed and operative in accordance with the present invention;

FIGS. 8A, 8B and 8C are schematic illustrations of the elements and function of an alternative embodiment to the navigator of FIG. 2, constructed and operative in accordance with the present invention;

FIGS. 9A and 9B are schematic illustrations of the elements of an alternative embodiment to the navigator of FIG. 2 at training and in operation, constructed and operative in accordance with the present invention; and

FIGS. 10A and 10B are schematic illustrations of the elements of an alternative embodiment to the navigator of FIGS. 9A and 9B at training and in operation, constructed and operative in accordance with the present invention.

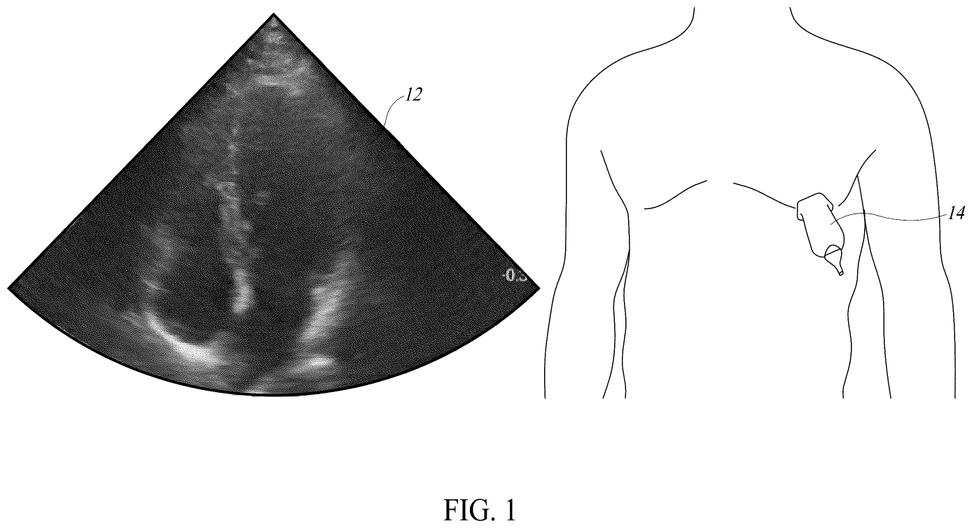

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Applicants have realized that the ability to use mobile ultrasound machines away from conventional places such as hospitals, means that untrained sonographers or non-sonographers might utilize these machines. However, untrained doctors, first aid providers or even patients themselves do not have the training or knowledge to administer these ultrasounds correctly. It will be appreciated that a different training is required for different organs and body parts.

Prior art systems such as that described in US Patent Publication No. US2018/0153505 entitled “Guided Navigation of an Ultrasound Probe”, published Jun. 7, 2018, and US Patent Publication No. US2016/0143627 entitled “Ultrasound Acquisition Feedback Guidance to a Target View”, published May 26, 2016, teach methodologies for determining the deviations between a supplied image and a preferred canonical image of a particular body part for helping the non-sonographer guide his or her transducer to the optimal orientation for capturing a best fit image.

Applicants have realized that these prior art systems do not provide a complete solution vis-a-vis rotation calculations. Applicants have also realized that these prior art systems are not particularly useful, since they require additional hardware (such as inertial measurement units such as magnetometers, gyroscopes, accelerometers etc.) to aid in determining the location of the non-sonographer's probe. Applicants have realized that a system which does not require additional hardware and which is easily accessible, such as via a download in order to be integrated or used as an overlay with the processing software of the pertinent mobile ultrasound machine, is far more usable. As a result, the present invention operates only with the digital images generated by the ultrasound unit.

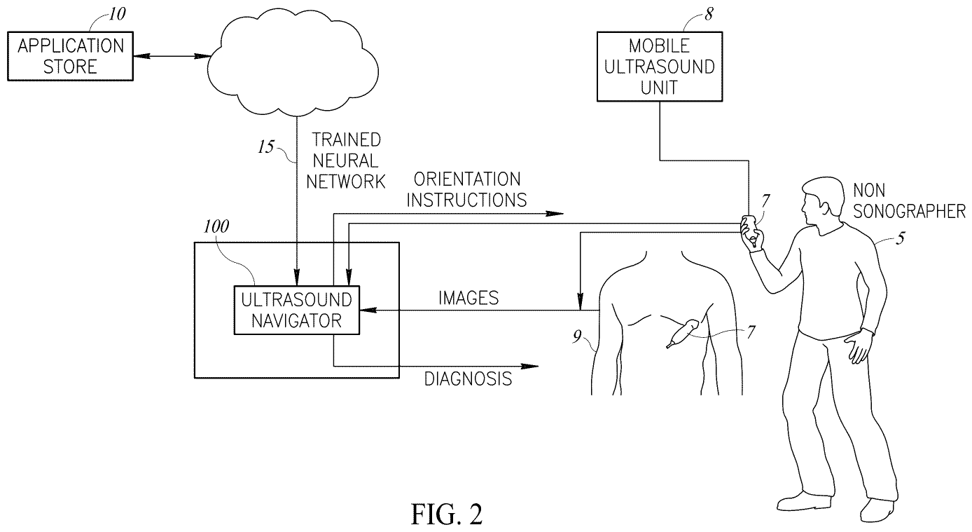

Reference is now made to FIG. 2 which illustrates an ultrasound navigator 100, according to a first embodiment of the present invention, which may be downloaded from a mobile application store 10, such as the Appstore of Apple or Google Play of Google, onto any portable computing device, such as a smartphone, a tablet, a laptop, a personal computer, a smart appliance, etc.

It will be appreciated that navigator 100 may comprise (as part of the download) a trained orientation neural network 15. Orientation neural network 15 is described in more detail herein below. As discussed herein above, navigator 100 may integrated or used as an overlay with processing software of the pertinent mobile ultrasound machine

Thus a user 5 may use a transducer or probe 7 (associated with mobile ultrasound unit 8) on patient 9 to supply images of a pertinent body part to navigator 100 and navigator 100 may supply orientation instructions accordingly as to how to orientate probe 7. It will be appreciated that the process may be iterative, with a non-sonographer or user 5 making more than one attempt to correctly orientate probe 7 in order to receive a suitable image. In accordance with a preferred embodiment of the present invention, “orientation” instructions may comprise both position (location in two or three-dimensional space) and rotation information (rotation in 3D space), even though navigator 100 receives only images.

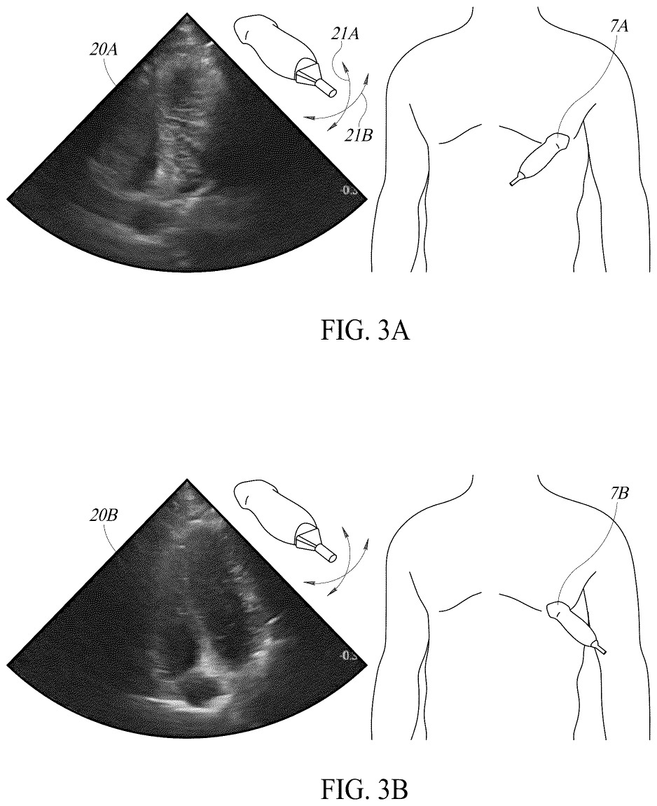

Reference is now made to FIGS. 3A and 3B which illustrate how navigator 100 may aid non-sonographer 5 to orientate probe 7 in order to capture a good image of a particular body part. FIG. 3A shows probe 7, labeled 7A, in the wrong position, i.e. the resultant image, labeled 20A, is not canonical. FIG. 3A additionally includes a set of arrows 21 instructing user 5 to change the rotation of probe 7A. Arrows 21A indicate a ‘pitch up’ kind of rotation. FIG. 3B shows probe 7B in the newly pitched US orientation and the resultant image 20B, which is better, though still not providing a canonical image. Arrows 21B indicate a new “yaw” rotation may be useful.

As discussed herein above, navigator 100 receives orientation neural network 15 which may be trained with expert data taken by a skilled sonographer for a particular body part or organ of interest. The training data received may include the canonical image of a particular body part as well as associated non-canonical images and for each, the orientation (i.e. position and rotation) of the sonographer's probe in space. It will be appreciated that this information may be generated using a probe with which an IMU (an inertial measurement unit which may include a magnetometer, a gyroscope, an accelerometer, etc.) is associated. The IMU may determine the orientation of the probe when an image is captured.

Reference is now made to FIG. 4 which illustrates the transformation between the orientation of a training probe 4c used by a trained sonographer for capturing the canonical image in relation to its orientation when capturing a non-canonical image for an organ. The orientation of training probe 4i when viewing the i^{th} non-canonical image may be defined as a “frame of reference” Fᵢ in space where frame of reference Fᵢ may have the six degrees of freedom (6DoF), corresponding to a three axis system (Q) having three rotations around the axes and three translations along the axes, that an IMU may measure.

Frames of reference Fᵢ may refer to frame of reference at an origin O, where, for the present invention, the origin may be at the organ and its frame of reference in space may be defined as Fₒ. For each frame of reference Fᵢ, there may be a transformation Rᵢ from the origin O, where the transformation Re may be a transformation to the desired orientation, labeled F_{c}, for viewing the canonical image, as follows:

R_{c}=F_{c}Fₒ⁻¹

Rᵢ=FᵢFₒ⁻¹ (1)

where Fₒ⁻¹ is the inverse transform of Fₒ. Thus, a transformation Tᵢ from the canonical pose to the i^{th} non-canonical pose may be RᵢR_{c}⁻¹:

Tᵢ=RᵢR_{c}⁻¹=FᵢFₒ⁻¹(FₒF_{c}⁻¹)=FᵢF_{c}⁻¹ (2)

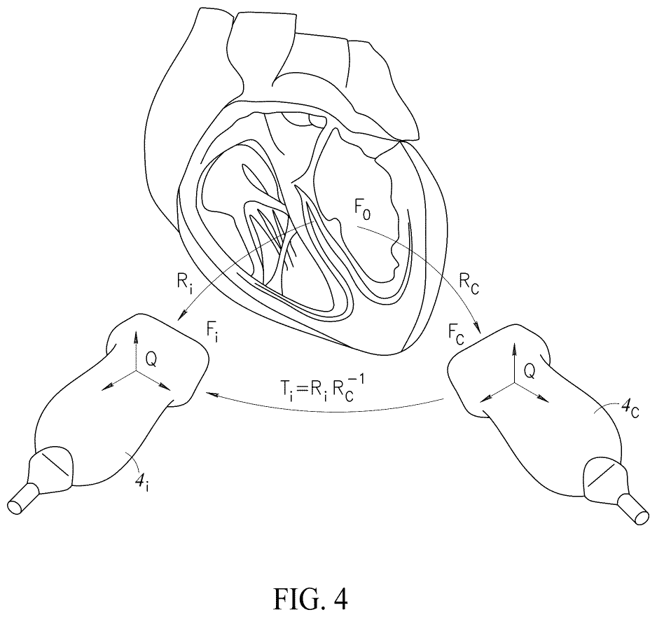

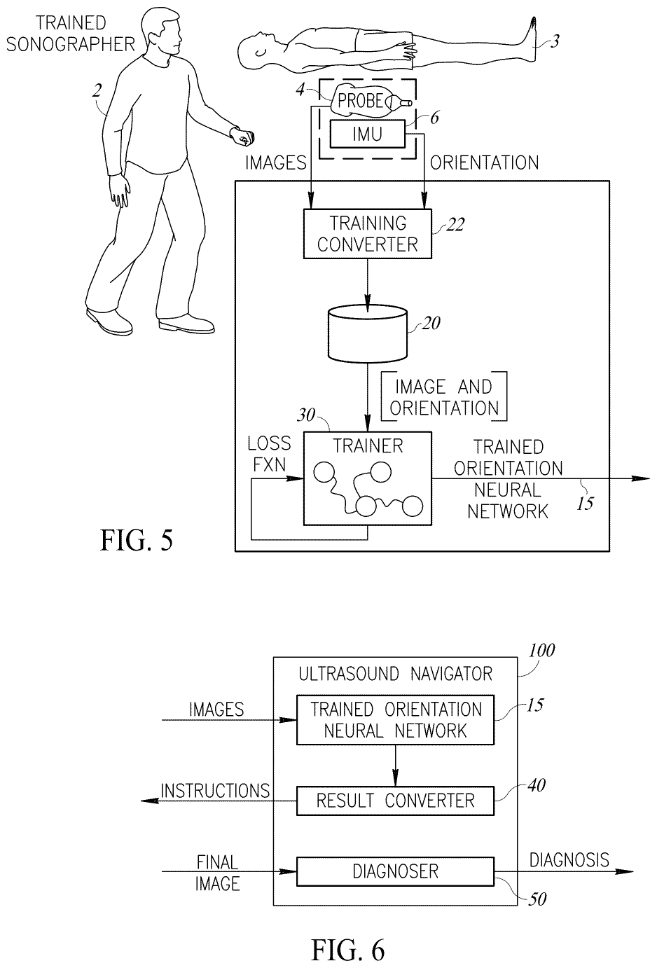

Reference is now made to FIG. 5 which illustrates the training process for orientation neural network 15 using a trainer 30. A skilled sonographer 2 using training probe 4 on a patient 3 may provide both canonical and associated non-canonical images for a particular body part. It will be appreciated that training probe 4 may be associated with an IMU 6 (an inertial measurement unit which may include a magnetometer, a gyroscope, an accelerometer, etc.) which may determine the orientation Fᵢ of the probe when an image is captured.

Training converter 22 may receive the orientation data Fᵢ for each image and may determine the transformation Tᵢ=RᵢR_{c}⁻¹ from the associated canonical position, as discussed herein above with respect to FIG. 4. Specifically, training converter 22 may take images X from training probe 4 and may process them as necessary. Database 20 may store non-canonical images Xᵢ together with their orientation data Fᵢ and their transformation data Tᵢ. Database 20 may also store canonical images X_{c} and their associated orientation data F_{c}. It will be appreciated that there may be multiple canonical images for a body part. For example, the heart has a four chamber canonical image, a two chamber canonical image, etc., and thus, training converter 22 may generate the transformation Tᵢ to each relevant canonical image. It will be appreciated that the relevant canonical image may be provided manually or determined automatically by any suitable algorithm.

It will be appreciated that the incoming training data to trainer 30 may be a combination of image Xᵢ and its associated ground truth transformation Tᵢ. For each non-canonical image, trainer 30 may learn the positioning transformation for the probe 4 to transform from viewing each canonical image to viewing each non-canonical image. It will be appreciated that the incoming data may comprise data from many different patients 3 so that trainer 30 may learn the changes in images Xᵢ, possibly due to the sex, age, weight, etc., of patient 3 and any other factors which may influence the transformation information between the non-canonical images and the canonical image.

It will be further appreciated that trainer 30 may be any suitable neural network trainer, such as a convolutional neural network trainer, which may train the network by updating the network to minimize an energy “loss” as determined by a loss function such as a distance between a calculated transformation S(Xᵢ) produced by orientation neural network 15 and the ground truth transformation Tᵢ for image Xᵢ from its associated canonical image. It will be appreciated that transformation S(Xᵢ) begins as an untrained neural network and finishes as a trained neural network.

The distance function may be any suitable distance function. If there is more than one associated canonical image, orientation neural network 15 may be trained with the ground truth transformation Tᵢ to each non-canonical image. A loss function “Loss” may be calculated as:

Loss=loss (S(Xᵢ),Tᵢ) (3)

Once orientation neural network 15 is trained, it may generate a transformation T for user probe 7 in response to each incoming image Xᵢ. This transformation may then be inverted or converted to guide user 5 from the orientation for the non-canonical image to the orientation for the canonical image, as described in more detail herein below.

Reference is now made to FIG. 6 which illustrates the components of navigator 100. Navigator 100 may comprise trained orientation neural network 15, a result converter 40 and a diagnoser 50.

As discussed herein above, user 5 may randomly place user probe 7 in relation to the desired body part. Trained orientation neural network 15 may provide the transformation T from the associated canonical image to the current non-canonical image of a particular body part. Result converter 40 may invert the generated transformation to provide orientation instructions for probe 7 from the current position and rotation viewing a non-canonical image to a position and rotation to view the associated canonical image. Result converter 40 may provide and display these orientation instructions to user 5 in various ways. It will be appreciated that this process may be iterative until user 5 positions probe 7 correctly (within an error range).

Result converter 40 may convert the orientation data S(X) produced by trained orientation neural network 15 into an explainable orientation for user 5, for a selected canonical image. Any suitable display may be utilized. An exemplary display is shown hereinabove with reference to FIGS. 3A and 3B. It will be appreciated that result converter 40 may use any appropriate interface and may (for example) display colored rotation markings. Moreover, result converter 40 may include elements that enable user 5 to indicate, when there are multiple canonical images for the body part, which canonical image is currently of interest.

Diagnoser 50 may receive the final canonical image produced by user 5 and may detect any anomalies therein. Diagnoser 50 may be any suitable diagnoser. For example, diagnoser 50 may implement the diagnosis method of PCT International Publication WO 2018/136805, published 26 Jul. 2018, assigned to the common assignees of the present invention, and incorporated herein by reference.

Applicants have realized that the fact that there are multiple canonical images for a single body part and the fact that there are standard, known motions from one canonical image to another may be utilized to reduce errors in the output of trained orientation neural network 15.

In this improved embodiment, orientation neural network 15 may be trained to the multiple canonical images. Thus, for each image Xᵢ, there may be multiple calculated transformations. For example, for a pair of canonical images c and c′, there may be a pair of calculated transformations S_{c}(Xᵢ) and S_{c′}(Xᵢ) for the same image Xᵢ which may have associated ground truth transformations T_{c,i} and T_{c′,i}.

Moreover, there is a known motion transformation Tₖ defined as:

Tₖ=R_{c}R_{c′}⁻¹ (4)

where R_{c} is for canonical image c and R_{c′} is for canonical image c′. These known motions are roughly constant across different subjects and therefore the transformation Tₖ from one canonical image c to another c′ may be utilized to constrain the calculated transformations S_{c}(Xᵢ) and S_{c′}(Xᵢ) to one of the canonical orientations. To do so, a probability measure Pₖ may be used to define a maximum likelihood loss term log Pₖ(S_{c}(Xᵢ)S_{c′}(Xᵢ)⁻¹) to add to the loss used to train orientation neural network 15, as follows:

Loss=loss(S_{c}(Xᵢ),T_{c,i})+loss(S_{c′}(Xᵢ),T_{c′i})−δ*log Pₖ(S_{c}(Xᵢ)S_{c′}(Xᵢ)⁻¹) (5)

The probability measure Pₖ may be determined experimentally by measuring the ground truth transformation Tₖ between canonical pose c and c′ across different subjects. Moreover, there may be multiple probability measures per body part, one for each pair of canonical images for the body part, and each probability measure Pₖ may define a separate additional term for the loss function.

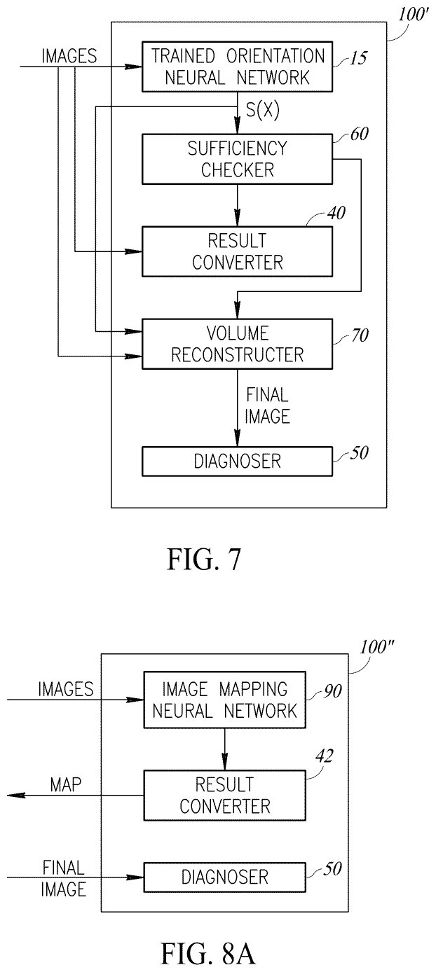

In an alternative embodiment, the navigator, here labeled 100′, may also comprise a sufficiency checker 60 and a volume reconstructer 70, as is illustrated in FIG. 7, to which reference is now made.

Volume reconstructer 70 may utilize the output of trained orientation neural network 15 and may produce 3D or 4D functions, and/or 3D volumes or 3D space-time volumes of the body parts of interest from the images Xᵢ produced by probe 7. In this embodiment, the images Xᵢ may be considered as cross-sections of the body part of interest.

Sufficiency checker 60 may check that sufficient cross sections have been received via trained orientation neural network 15 in order to perform the 3D/4D volume reconstruction and may guide user 5 (via result converter 40) accordingly. For example, sufficiency checker 60 may determine when a pre-defined minimal number of images have been taken.

Upon an indication from sufficiency checker 60, volume reconstructer 70 may generate the 3D/4D volume, after which, reconstructer 70 may pull the relevant canonical views from the generated volume and may provide them to diagnoser 50. It will be appreciated that the canonical views in this embodiment are produced from the generated volume and may or may not have been among the images used to produce the volume.

Volume reconstructer 70 may utilize tomographic reconstruction, such as that based on inverse Radon transformation or other means, to reconstruct the 3D/4D functions and/or volumes from the images. It will be appreciated that for successful volumatic tomographic reconstruction, it is crucial to know the cross-section's position in 3D space or 4D space-time. Applicants have realized that trained orientation neural network 15 may provide a suggested transformation S(X) for probe 7 for each image taken and that transformation S(X) may be used to rotate the pixels of image Xᵢ from a fixed 2D imaging plane to the 3D orientation Q in space in which probe 4ᵢ was positioned when it produced image Xᵢ.

Volume reconstructer 70 may receive the transformation S(Xᵢ) from trained orientation neural network 15 for each image Xᵢ and may apply the transformation to move the image from an imaging plane (as output from the probe) to a plane defined by the transformation of the probe, producing a rotated cross-section CSᵢ of the body part. Volume reconstructer 70 may then use tomographic reconstruction to build the volume of the body part of interest from the images cross-sections CS(Xᵢ).

To apply transformation S(Xᵢ), it will first be appreciated that image Xᵢ comprises a set of pixels having a 2D location (xⱼ,yⱼ) within the 2D imaging plane and an intensity Iⱼ. Volume reconstructer 70 may apply transformation S(Xᵢ) on a 3D pixel location (xⱼ,yⱼ,0) in space to generate an approximation of the 3D orientation Q of image Xᵢ, after which it may apply an operator H to center or scale the orientated image Xᵢ, as follows:

Q=H*S(Xᵢ)*[xⱼ,yⱼ,0]^{T} (6)

Volume reconstructer 70 may provide the generated canonical image to diagnoser 50 which may then produce a diagnosis from it, as described hereinabove.

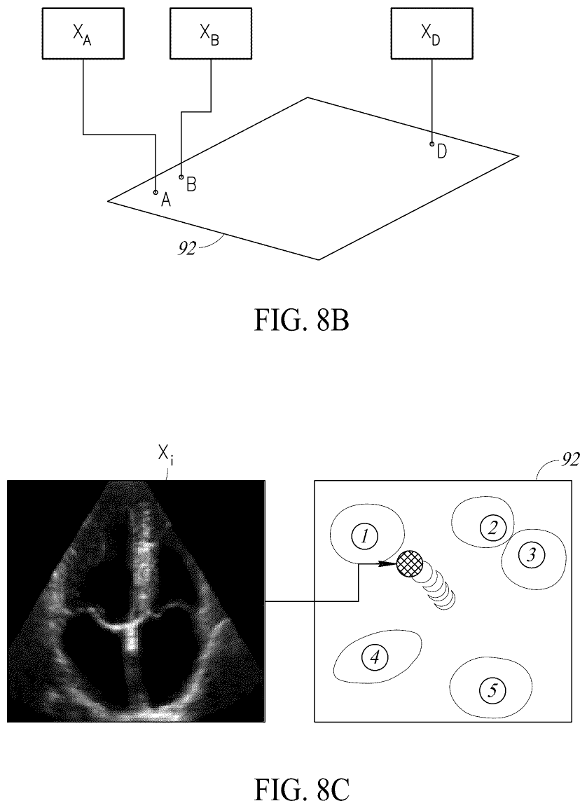

In yet another embodiment, illustrated in FIGS. 8A, 8B and 8C to which reference is now made, navigator, here labeled 100″, may comprise an image mapping neural network 90. Mapping neural network 90 may map each image Xᵢ onto a 2D plane 92 (FIG. 8B). FIG. 8B shows three exemplary images X_{A}, X_{B} and X_{D} being mapped to three different locations A, B and D on plane 92.

Result converter, here labeled 42, may display 2D plane 92 to user 5, marking his current location in one color (for example, as a grey dot (shown in FIG. 8C as a shaded dot)) and the location of the canonical images for this body part as dots of other colors (shown in FIG. 8C as numbered circles 1-5). FIG. 8C also shows the acquired image Xᵢ and its map 92. Map point M(Xᵢ) may represent non-canonical image Xᵢ on map 92 and the other numbered circles may be canonical map points representing the desired or required canonical views c. User 5 may use trial and error movements of probe 7 to move map point M(Xᵢ) nearer towards the desired circles and mapper 90 may regenerate 2D plane 92 for each new image i from probe 7.

Applicants have realized that small changes in the motion of probe 7 should generate small motions on 2D plane 92 and that distances between images Xᵢ should be similar to the distance between map locations. Applicants have further realized that optimal paths from one canonical image to another should be straight, constant speed trajectories.

It will be appreciated that for this embodiment, mapping neural network 90 may be trained using incoming data which may include each image Xᵢ and the image Xe of its associated canonical view.

Mapping neural network 90 may incorporate a loss function to minimize a distance between a calculated map point M(Xᵢ) currently produced by neural network 90 during training and the associated map point M(X_{c}) for each canonical view cⱼ:

Loss=loss(M(Xᵢ),M(X_{cj})) (7)

To incorporate an optimal path to the different canonical views, a probability vector p_{i,j} may be added which may define how close the image Xᵢ is on a path to the jth desired canonical image c. The loss function may then be updated to be:

Loss=loss(M(Xᵢ),Σp_{i,j}M(X_{cj})) (8)

To preserve distances, the loss function may be updated to be:

Loss=loss(M(Xᵢ),Σp_{i,j}M(X_{cj}))+loss(dist(Xᵢ,Xⱼ),∥M(Xᵢ)−M(Xⱼ)∥₂) (9)

It will be appreciated that plane 92 may be either a 2D plane or a 3D volume, as desired. The mapping operations discussed herein above are operative for mapping to a 3D volume as well.

Applicants have realized that neural networks can be trained not just to generate transformation information but to generate canonical images, given the right kind of training. This might be particularly useful if the input from non-sonographers is expected to be noisy (since they may not have steady enough hands) and/or if it is desired to see, at the canonical view, the body part functioning. For example, ultrasound sonographers regularly provide information about a full cardiac cycle, from systole to distole and back to systole, for cardiac function analysis.

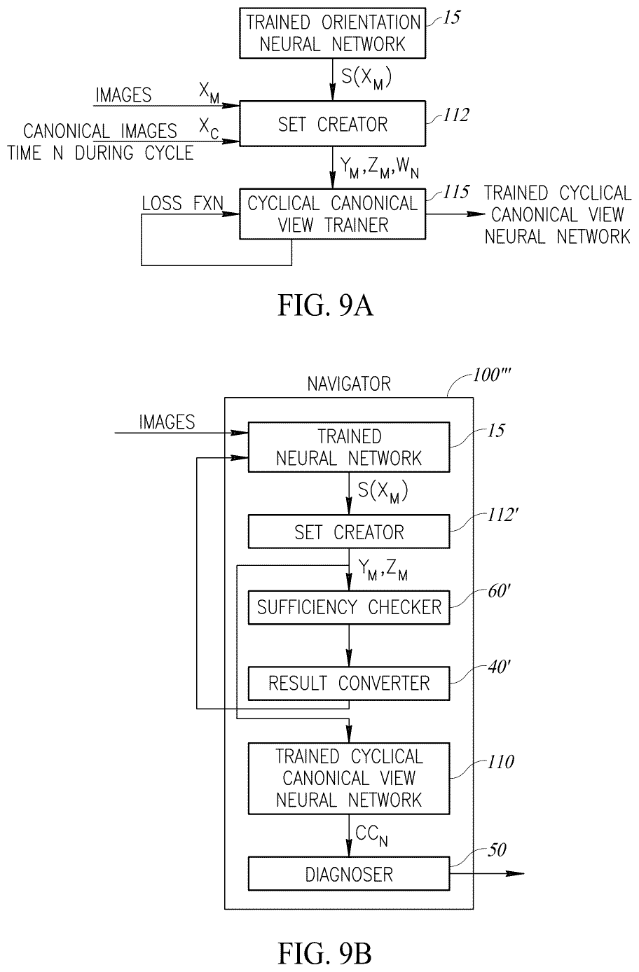

In yet another embodiment, shown in FIGS. 9A and 9B to which reference is now made, navigator 100 may comprise a cyclical canonical view neural network 110, which may be a neural network trained from the output of trained orientation neural network 15. Canonical view cycler 110 may aggregate repeating images to reduce noise and to provide a less noisy summarization of (for example) an organ cycle, such as the cardiac cycle.

As shown in FIG. 9A, the elements needed for training cyclical canonical view neural network 110 may comprise trained orientation neural network 15, a set creator 112 to create the input to network 110, and a cyclical canonical view trainer 115.

For this embodiment, skilled sonographer 2 may provide multiple ultrasound images m taken over time as well as multiple images n taken over time at one canonical view pose c. Set creator 112 may receive image Xₘ from trained orientation neural network 15 along with its associated transformation information S(Xₘ) and may combine these with their associated image X_{c,n} taken at the canonical view. Skilled sonographer 2 may provide such associations.

Set creator 112 may then generate triplets {[Yₘ, Zₘ], Wₙ} where [Yₘ, Zₘ] are input to cyclical canonical view trainer 115 and Wₙ is the associated output. Each Yₘ may consist of a set of g images where Yₘ={X₁, X₂, . . . X_{g}} and Zₘ may consist of the transformation information S(X) of the images Yₘ such that Zₘ={S(X₁), S(X₂) . . . . S(X_{g})}. Typically, g may be 10-100 images.

Each pair [Yₘ,Zₘ] may have a set Wₙ of associated canonical images X_{c} taken at the canonical view c at times between 0 and n. The time n may indicate the time within the cardiac cycle. As mentioned herein above, skilled sonographer 2 may indicate the cardiac cycle information and may provide the associated canonical images X_{c} which will be included in set Wₙ.

In this scenario, cyclical canonical view trainer 115 may receive as input general frames Yₘ, their approximate transformations Zₘ as generated by orientation neural network 15, and their associated cardiac cycle timing n, and may be trained to generate a set of summary images Wₙ in a canonical view at desired times n. The optimization is:

Loss=loss(CCₙ,Wₙ) (10)

where CCₙ is the output of the cyclical canonical view neural network 110 as it is being trained.

Cyclical canonical view trainer 115 may generate trained cyclical canonical view neural network 110 for navigator 100 using any appropriate neural network, such as a fully-convolutional network, an encoder-decoder type of network or a generative adversarial network.

As illustrated in FIG. 9B to which reference is now made, navigator 100″′ may comprise trained orientation neural network 15, a set creator 112′ for operation, a sufficiency checker 60′, a result converter 40′, trained cyclical canonical view neural network 110 and diagnoser 50.

In operation, non-sonographer 5 may operate probe 7 near the body part of interest over a period of time, at least long enough to cover the desired body part cycle (such as the cardiac cycle). The images from probe 7 may be provided to trained orientation neural network 15 to generate their associated transformations S(X) and to set creator 112′ to generate the appropriate sets Yₘ and Zₘ. Sufficiency checker 60′ may check that sets Yₘ and Zₘ are large enough and may instruct result converter 40′ to instruct user 5 either to orientate probe 7 in a desired way or to continue viewing at the current orientation. It will be appreciated that, in this embodiment, non-sonographer 5 does not have to hold probe 7 at exactly the canonical view and thus, the instructions that result converter 40′ may provide may be coarser. Cyclical canonical view neural network 110 may generate the summary cyclical, canonical views CCₙ from the output of set creator 112′.

It will be appreciated that this embodiment may also be useful for non-cyclical body parts, particularly for when user 5 may hold probe 7 unsteadily. In this embodiment, each set may have only one or two images therein.

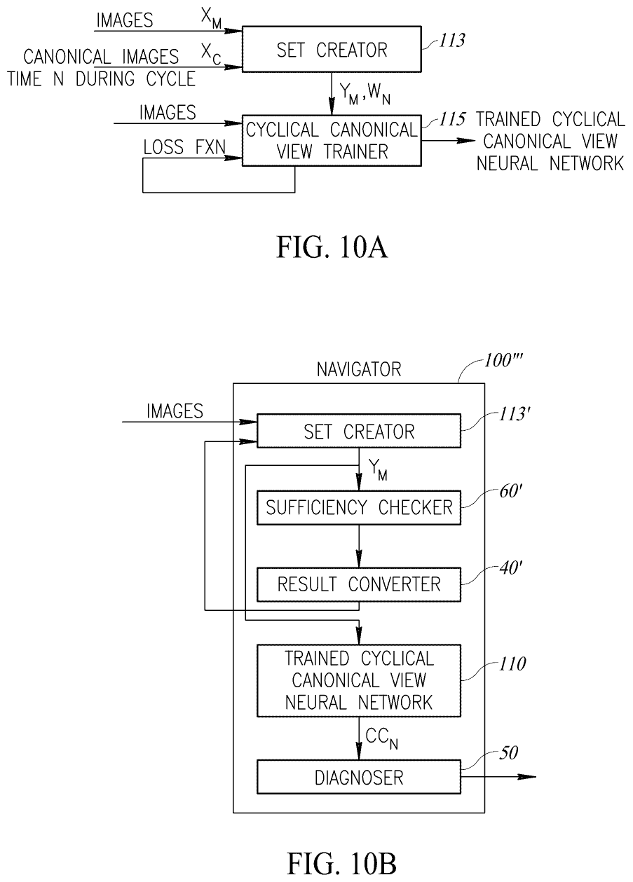

Applicants have further realized that neural networks can also be trained without the transformation information produced by trained orientation neural network 15. This is shown in FIGS. 10A and 10B, which illustrate a system similar to that of FIGS. 9A and 9B, but without trained orientation neural network 15. As a result, for training (FIG. 10A) a set creator 113 may create Yₘ from images Xᵢ and may create Wₙ from canonical images X_{c} at times n. Cyclical canonical view trainer 115 may generate cyclical canonical view neural network 110 using equation (10).

At runtime (FIG. 10B), a set creator 113′ may create Yₘ from images Xᵢ and cyclical canonical view neural network 110 may generate the summary views CCₙ.

It will be appreciated that the present invention may provide a navigator for non-sonographers to operate a mobile ultrasound machine without training and without any additional hardware other than the ultrasound probe. Thus, the navigator of the present invention receives ultrasound images as its only input. It will further be appreciated that this may enable non-sonographers to perform ultrasound scans in many non-conventional scenarios, such as in ambulances, in the battlefield, at urgent care facilities, nursing homes etc.

Moreover, the present invention may be implemented in more conventional scenarios, such as part of conventional machines used in hospital or clinic environments, which may also be implemented on carts.

Unless specifically stated otherwise, as apparent from the preceding discussions, it is appreciated that, throughout the specification, discussions utilizing terms such as “processing,” “computing,” “calculating,” “determining,” or the like, refer to the action and/or processes of a general purpose computer of any type such as a client/server system, mobile computing devices, smart appliances or similar electronic computing device that manipulates and/or transforms data represented as physical, such as electronic, quantities within the computing system's registers and/or memories into other data similarly represented as physical quantities within the computing system's memories, registers or other such information storage, transmission or display devices.

Embodiments of the present invention may include apparatus for performing the operations herein. This apparatus may be specially constructed for the desired purposes, or it may comprise a general-purpose computer or a client/server configuration selectively activated or reconfigured by a computer program stored in the computer. The resultant apparatus when instructed by software may turn the general purpose computer into inventive elements as discussed herein. The executable instructions may define the inventive device in operation with the computer platform for which it is desired. Such a computer program may be stored in a computer accessible storage medium which may be a non-transitory medium, such as, but not limited to, any type of disk, including optical disks, magnetic-optical disks, read-only memories (ROMs), volatile and non-volatile memories, random access memories (RAMs), electrically programmable read-only memories (EPROMs), electrically erasable and programmable read only memories (EEPROMs), magnetic or optical cards, Flash memory, disk-on-key or any other type of media suitable for storing electronic instructions and capable of being coupled to a computer system bus.

The processes and displays presented herein are not inherently related to any particular computer or other apparatus. Various general-purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the invention as described herein.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A computer-implemented method for providing real-time visual guidance for orienting an ultrasound probe toward a canonical ultrasound view, the method comprising:
receiving successive ultrasound images acquired by an ultrasound probe during a scanning procedure, each ultrasound image reflecting a current probe position and rotation relative to an anatomical region;
a trained neural network, generating from said received ultrasound images, without reliance on external position or motion sensors, a spatial mapping which encodes one or more adjustments in probe position and rotation required to achieve a target spatial pose associated with the canonical ultrasound view;
transforming the spatial mapping into visual guidance data to achieve the required adjustment;
generating and displaying, on a graphical user interface, one or more visual guidance elements derived from the visual guidance data to instruct a user how to manipulate the probe; and
updating the visual guidance elements until the canonical ultrasound view is achieved.

2. The method of claim 1, wherein the one or more visual guidance elements comprise a set of arrows, and wherein the type of rotation visually communicated is one of a pitch or a yaw.

3. The method of claim 1, wherein the one or more visual guidance elements comprises colored rotation markings.

4. The method of claim 1, wherein the updating is performed until the current probe orientation is within a predefined error range of the target probe orientation.

5. The method of claim 1, wherein the graphical user interface provides an instruction to the user to either move the ultrasound probe or to continue viewing at the current probe orientation.

6. The method of claim 1, wherein the updating step generates updated visual guidance for each new subsequent ultrasound image captured by the probe.

7. The method of claim 1, wherein the trained orientation neural network was trained using a training dataset comprising at least one canonical view, a plurality of non-canonical images captured around the at least one canonical view, and corresponding ground-truth transformations, each ground-truth corrective orientation representing a required adjustment in position and rotation between a non-canonical image and the canonical view.

8. The method of claim 7, wherein the ground-truth corrective orientations were determined by from inertia measurement unit (IMU) data received from an IMU mounted on a training probe during a training session.

9. The method of claim 7, wherein the training comprises minimizing a loss function, the loss function configured to reduce a distance between a calculated corrective orientation produced by the orientation neural network and the corresponding ground-truth corrective orientation for each non-canonical image.

10. The method of claim 9, wherein the loss function further includes an additional term based on a probability measure to constrain the calculated transformation relative to known motions between a plurality of different canonical views.

11. The method of claim 1, wherein the canonical view is one of a plurality of available canonical views for a body part.

12. The method of claim 1, further comprising: upon the current probe orientation reaching the target probe orientation, making a diagnosis from the current ultrasound image.

13. The method of claim 1, wherein the computing device is one of: a smartphone, a tablet, a laptop, a personal computer, or a smart appliance.
